(19) United States
(12) Patent Application Publication
(10) Pub. No.: US 2026/0166103 A1
(43) Pub. Date: Jun. 18, 2026

(54) CANNABIGEROLIC ACID (CDGA) DERIVATIVES AND USES THEREOF

(71) Applicants: EPM (IP), INC., San Francisco, CA (US); YISSUM RESEARCH DEVELOPMENT COMPANY OF THE HEBREW UNIVERSITY OF JERUSALEM LTD., Jerusalem (IL)

(72) Inventors: Reshef Swisa, Santa Monica, CA (US); Anat Iosub-Amir, Jerusalem (IL); Ron Shaharabani, Herzliya (IL); Raphael Mechoulam, Jerusalem (IL); Natalya M. Kogan, Kfar Adummim (IL)

(21) Appl. No.: 19/529,854

(22) Filed: Feb. 4, 2026

Related U.S. Application Data
(63) Continuation of application No. 18/000,201, now abandoned.
(60) Provisional application No. 63/033,851, filed on Jun. 3, 2020.

(51) Int. Cl. A61K 36/185; A61K 31/05; A61K 31/192; A61K 45/06; A61P 25/08; A61P 29/00; C07C 65/28; C07C 69/92

(52) U.S. Cl. CPC A61K 36/3482; A61K 31/05; A61K 31/192; A61K 45/06; A61P 25/08; A61P 29/00; C07C 65/28; C07C 69/92

(57) ABSTRACT

The present invention provides novel compounds comprising cannabigerlic acid (CBGA) derivatives, including acids and esters, and uses thereof in the treatment of various diseases, disorders, conditions and symptoms.

### CROSS-REFERENCE TO RELATED APPLICATIONS

This application is a continuation of U.S. application Ser. No. 18/000,201 filed on Nov. 29, 2022, which is a National Stage Application from International Application No. PCT/IL2021/050663 filed on Jun. 3, 2021, which claims priority to U.S. Provisional Patent Application No. 63/033,851 filed on Jun. 3, 2020, the disclosure of each of which is incorporated herein, in its entirety, by this reference.

### FIELD OF THE INVENTION

The present invention relates to cannabigerolic acid (CBGA) derivatives, including acids and esters, and uses thereof in the treatment of various diseases, disorders, conditions and symptoms.

### BACKGROUND OF THE INVENTION

Cannabigerol (CBG) is one of more than 120 identified cannabinoid compounds found in the plant genus Cannabis. Cannabigerol is the non-acidic form of cannabigerolic acid (CBGA), the parent molecule from which other cannabinoids are synthesized. Cannabigerol is a minor constituent of cannabis. Cannabigerolic acid (CBGA) is a natural non-psychoactive precursor of CBG. During growth, most of the cannabigerol is converted into other cannabinoids, primarily tetrahydrocannabinol (THC) or cannabidiol (CBD), leaving about 1% cannabigerol in the plant.

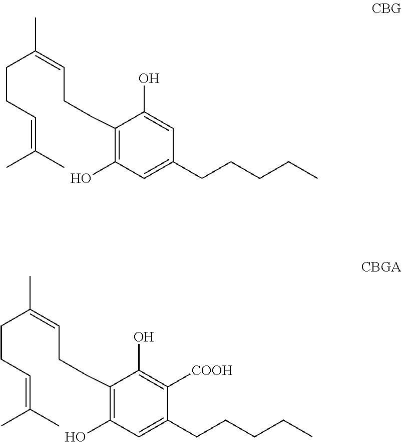 [SMILES: CCCCCc1cc(O)c(CC/C=C(/C)CCC=C(C)C)c(O)c1 ; CCCCCc1cc(O)c(CC/C=C(/C)CCC=C(C)C)c(O)c1C(=O)O]

Mechoulam and Gaoni (Tetrahedron., 1965, 21, pp. 1223-1229) disclose the isolation and structure of cannabinolic, cannabidiolic and cannabigerolic acids.

Crombie et al. (Phytochemistry., 1977, Vol. 16, p. 1413) disclose miniaturized synthesis and chromatographic study of cannabinoid acids and esters.

Ahmed et al. (J Nat Prod., 2008 April, 71(4), pp. 536-542) disclose the isolation of eleven cannabinoid esters, together with three cannabinoid acids and Δ⁹-tetrahydrocannabinol, from a high-potency variety of Cannabis sativa.

WO 2018/235079 discloses compositions comprising CBDA esters and uses thereof in the treatment of a condition, disease or symptom associated with 5-HT_{1A} receptor.

US Patent application 2003/0232101 and U.S. Pat. No. 7,105,685 disclose methods of treatment of diseases associated with immune dysfunction, particularly HIV related disease and neoplastic disorders, by administering pharmacologically-acceptable compositions, which inter alia comprise different CBGA ester derivatives.

US Patent application 2010/0298579 discloses processes for the preparations of different cannabinoids through decarboxylation of the corresponding cannabinoid acids.

There is still an unmet medical need for effective therapies for use of non-psychoactive cannabinoids, particularly derivatives of CBG with improved properties and decreased potential for detrimental activity. These derivatives will be advantageous in the treatment of various diseases, conditions, disorders and symptoms, such as pain, impaired neurological functions including depression and anxiety, and inflammation.

### SUMMARY OF THE INVENTION

The present invention provides novel compounds comprising cannabigerolic acid (CBGA) derivatives, including acid and esters, and uses thereof. The present invention further provides pharmaceutical compositions comprising CBGA derivatives, alone or in combination with one or more additional cannabinoid compounds, for use in treating various diseases and disorders and symptoms, including but not limited to pain, impaired neurological function, inflammation including respiratory inflammation, inflammatory bowel disease and autoimmune diseases, nausea, vomiting, convulsions, low appetite and glaucoma.

It is now disclosed, unexpectedly, that compositions comprising cannabigerolic acid (CBGA) or CBGA ester derivatives exhibit prolonged and significant therapeutic effects compared to cannabigerol (CBG). Advantageously, the pharmaceutical compositions disclosed herein are non-psychoactive. According to some embodiments the present invention provides pharmaceutical compositions comprising CBGA derivatives having superior pharmacokinetics compared to the commercially available cannabigerol (CBG).

According to one aspect, the present invention provides a compound represented by the structure of Formula (I),

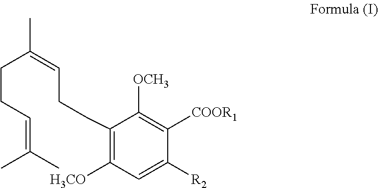

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, R₁ is hydrogen. According to some embodiments, R₁ is a linear unsubstituted C₁-C₁₅ alkyl. According to some embodiments, R₁ is a linear substituted C₁-C₁₅ alkyl. According to some embodiments, R₁ is a branched unsubstituted C₁-C₁₅ alkyl. According to some embodiments, R₁ is a branched substituted C₁-C₁₅ alkyl. According to some embodiments, R₁ is methyl. According to some embodiments, R₁ is a linear unsubstituted C₂-C₁₅ alkenyl. According to some embodiments, R₁ is a linear substituted C₂-C₁₅ alkenyl. According to some embodiments, R₁ is a branched unsubstituted C₂-C₁₅ alkenyl. According to some embodiments, R₁ is a branched substituted C₂-C₁₅ alkenyl. According to some embodiments, R₁ is a linear unsubstituted C₂-C₁₅ alkynyl. According to some embodiments, R₁ is a linear substituted C₂-C₁₅ alkynyl. According to some embodiments, R₁ is a branched unsubstituted C₂-C₁₅ alkynyl. According to some embodiments, R₁ is a branched substituted C₂-C₁₅ alkynyl.

According to some embodiments, R₂ is a linear unsubstituted C₁-C₁₅ alkyl. According to some embodiments, R₂ is a linear substituted C₁-C₁₅ alkyl. According to some embodiments, R₂ is a branched unsubstituted C₁-C₁₅ alkyl. According to some embodiments, R₂ is a branched substituted C₁-C₁₅ alkyl. According to some embodiments, R₂ is a linear unsubstituted C₅ alkyl (n-pentyl). According to some embodiments, R₂ is a linear unsubstituted C₂-C₁₅ alkenyl. According to some embodiments, R₂ is a linear substituted C₂-C₁₅ alkenyl. According to some embodiments, R₂ is a branched unsubstituted C₂-C₁₅ alkenyl. According to some embodiments, R₂ is a branched substituted C₂-C₁₅ alkenyl. According to some embodiments, R₂ is a linear unsubstituted C₂-C₁₅ alkynyl. According to some embodiments, R₂ is a linear substituted C₂-C₁₅ alkynyl. According to some embodiments, R₂ is a branched unsubstituted C₂-C₁₅ alkynyl. According to some embodiments, R₂ is a branched substituted C₂-C₁₅ alkynyl.

According to some embodiments, the compound is represented by the structure of Formula (II),

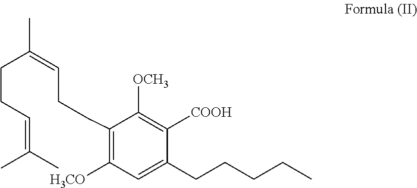 [SMILES: CCCCCc1cc(OC)c(CC/C=C(/C)CCC=C(C)C)c(OC)c1C(=O)O]

and stereoisomers, geometrical, and salts thereof.

According to some embodiments, the compound is represented by the structure of Formula (III) (designated herein compound 1),

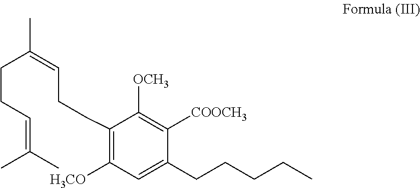 [SMILES: CCCCCc1cc(OC)c(CC/C=C(/C)CCC=C(C)C)c(OC)c1C(=O)OC]

and stereoisomers, geometrical, and salts thereof.

According to another aspect, the present invention provides a pharmaceutical composition comprising a therapeutically effective amount of a compound represented by the structure of Formula (I) as defined herein above, and a pharmaceutically acceptable carrier or excipient.

According to some embodiments, the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a preservative, an antioxidant, a flavoring agent, a colorant, and a mixture or combinations thereof.

According to some embodiments, the pharmaceutical composition is in a form selected from the group consisting of tablet, pill, capsule, pellets, granules, powder, a wafer, coated or uncoated beads, lozenge, sachet, cachet, elixir, a depot system, a patch, suspension, dispersion, emulsion, solution, syrup, aerosol, oil, ointment, suppository, a gel, and a cream.

According to some embodiments, the pharmaceutical composition is formulated into a dosage form suitable for, oral, intranasal, intravenous, intraarterial, topical, intramuscular, transdermal, intraperitoneal, intrathecal, vaginal, rectal or subcutaneous administration.

According to some embodiments, the pharmaceutical composition is for use in the treatment of a disease, disorder or symptom amenable to treatment with cannabigerol (CBG).

According to another aspect, the present invention provides a pharmaceutical composition comprising a therapeutically effective amount of a compound represented by the structure of Formula (I), for use in the treatment of a disease, disorder or symptom amenable to treatment with

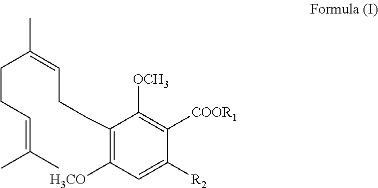

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, R₁ is hydrogen. According to some embodiments, R₁ is a linear unsubstituted C₁-C₁₅ alkyl. According to some embodiments, R₁ is methyl. According to some embodiments, R₂ is a linear unsubstituted C₁-C₁₅ alkyl. According to some embodiments, R₂ is a linear unsubstituted C₅ alkyl.

According to some embodiments, the disease, disorder or symptom amenable to treatment with CBG is selected from the group consisting of pain, autoimmune disease, cancer, bacterial infection, impaired neurological function, inflammation, nausea, vomiting, convulsions, low appetite and glaucoma.

According to some embodiments, the pharmaceutical composition is for use in the treatment of pain.

According to some embodiments, the pharmaceutical composition is for use in the treatment of autoimmune disease.

According to some embodiments, the pharmaceutical composition is for use in the treatment of cancer.

According to some embodiments, the pharmaceutical composition is for use in the treatment of bacterial infection.

According to some embodiments, the pharmaceutical composition is for use in the treatment of impaired neurological function.

According to some embodiments, the impaired neurological function is selected from the group consisting of stroke, trauma, Parkinson's Disease, vascular dementia, senile dementia, Alzheimer's disease, mild cognitive impairment, Huntington's Disease, Amyotrophic lateral sclerosis (ALS), epilepsy, multiple sclerosis, and psychiatric disorders. Each possibility represents a separate embodiment of the present invention.

According to some embodiments, the impaired neurological function is epilepsy.

According to some embodiments, the impaired neurological function is a psychiatric disorder.

According to some embodiments, the psychiatric disorder is selected from the group consisting of depression, anxiety, acute or chronic stress, schizophrenia, panic attacks, ADHD, bipolar disorder, obsessive-compulsive disorder and personality disorders. Each possibility represents a separate embodiment of the present invention.

According to some embodiments, the pharmaceutical composition is for use in the treatment of an inflammation. According to some embodiments, the pharmaceutical composition is for use in treating joint inflammatory diseases and joint degeneration. According to additional embodiments, the pharmaceutical composition is for use in treating respiratory inflammation.

According to additional embodiments, the pharmaceutical composition is for use in treating inflammatory bowel disease.

According to some embodiments, the pharmaceutical composition is for use in the treatment of nausea, vomiting or low appetite.

According to some embodiments, the pharmaceutical composition is for use in the treatment of convulsions.

According to some embodiments, the pharmaceutical composition is for use in the treatment of glaucoma.

According to some embodiments, the pharmaceutical composition is for use in the treatment of gastrointestinal diseases or disorders.

According to some embodiments, the disease, disorder or symptom amenable to treatment with CBG is selected from the group consisting of Non-Alcoholic Fatty Liver Disease (NAFLD), chronic kidney disease (CKD), obesity, hyperglycemia, diabetes, metabolic syndrome and/or obesity related diseases.

According to some embodiments, the pharmaceutical composition is for use in the treatment of obesity.

According to some embodiments, the pharmaceutical composition is for use in the treatment of muscular dystrophy.

According to some embodiments, the compound is represented by the structure of Formula (II),

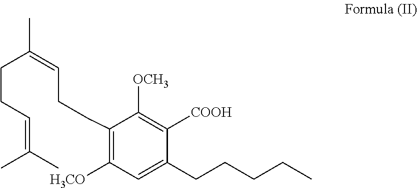 [SMILES: CCCCCc1cc(OC)c(CC/C=C(/C)CCC=C(C)C)c(OC)c1C(=O)O]

and stereoisomers, geometrical, and salts thereof.

According to some embodiments, the compound is represented by the structure of Formula (III) (designated herein EPM 310),

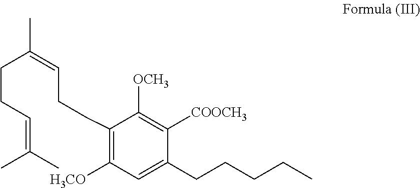 [SMILES: CCCCCc1cc(OC)c(CC/C=C(/C)CCC=C(C)C)c(OC)c1C(=O)OC]

and stereoisomers, geometrical, and salts thereof.

According to some embodiments, the pharmaceutical composition further comprising at least one additional active pharmaceutical ingredient.

According to some embodiments, the additional active pharmaceutical ingredient comprises at least one additional cannabinoid compound.

According to some embodiments, the additional cannabinoid compound is selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), esters thereof and combinations thereof.

According to some embodiments, the additional cannabinoid compound comprises a cannabigerolic acid (CBGA) ester represented by Formula (IV), a cannabinolic acid (CBNA) ester represented by Formula (V), a cannabidiolic acid (CBDA) ester represented by Formula (VI), a tetrahydrocannabinolic acid (THCA) ester represented by Formula (VII) or any combination thereof,

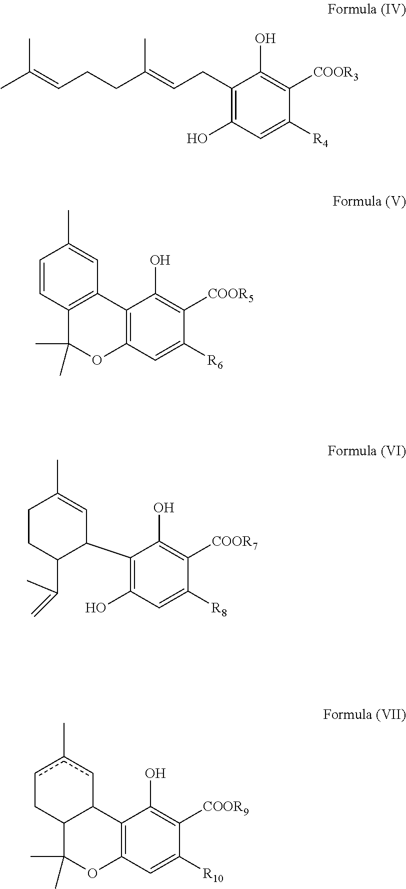

wherein
each of R₃, R₄, R₅, R₆, R₇, R₈, R₉ and R₁₀ is independently selected from the group consisting of a linear or branched, unsubstituted or substituted C₁-C₁₅ alkyl, a linear or branched, unsubstituted or substituted C₂-C₁₅ alkenyl, and a linear or branched, unsubstituted or substituted C₂-C₁₅ alkynyl;
one of the dashed lines in Formula (VII) represents a double bond and the other dashed line represents a single bond;
and stereoisomers, geometrical, and salts thereof. Each one of the cannabigerolic acid (CBGA) ester represented by Formula (IV), the cannabinolic acid (CBNA) ester represented by Formula (V), the cannabidiolic acid (CBDA) ester represented by Formula (VI), and the tetrahydrocannabinolic acid (THCA) ester represented by Formula (VII) represents a separate embodiment of the present invention.

According to some embodiments, the at least one additional cannabinoid compound is cannabidiolic acid methyl ester (CBDA-ME).

According to some embodiments, the at least one additional cannabinoid compound is present in one or more extracts of a cannabis plant. According to some embodiments, the cannabis plant extract is obtained from a strain selected from the group consisting of Cannabis sativa, Cannabis indica, Cannabis ruderalis, a hybrid strain, and combinations thereof. According to some embodiments, the cannabis plant extract is obtained from a strain selected from the group consisting of a high-CBD strain, a high-THC strain, and a combination thereof.

According to some embodiments, the cannabis plant extract comprises at least one cannabinoid selected from the group consisting of cannabidiol (CBD), tetrahydrocannabinol (THC), cannabinol (CBN), cannabigerol (CBG), cannabichromene (CBC), acids thereof and combinations thereof.

According to some embodiments, the cannabis plant extract comprises from about 1 to about 25% (w/w) of CBD. According to some embodiments, the cannabis plant extract comprises about 1% (w/w) of CBD. According to some embodiments, the cannabis plant extract comprises about 10% (w/w) of CBD. According to some embodiments, the cannabis plant extract comprises about 25% (w/w) of CBD.

According to some embodiments, the cannabis plant extract comprises from about 1 to about 25% (w/w) of THC. According to some embodiments, the cannabis plant extract comprises about 1% (w/w) of THC. According to some embodiments, the cannabis plant extract comprises about 10% (w/w) of THC. According to some embodiments, the cannabis plant extract comprises about 25% (w/w) of THC.

According to another aspect, the present invention provides a method of treating a disease, disorder or symptom amenable to treatment with cannabigerol (CBG), the method comprising the step of administering to a subject in need thereof a pharmaceutical composition comprising a therapeutically effective amount of a compound represented by the structure of Formula (I), and a pharmaceutically acceptable carrier or excipient,

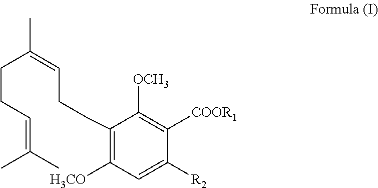

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, wherein R₁ is a hydrogen. According to some embodiments, wherein R₁ is a linear unsubstituted C₁-C₁₅ alkyl. According to some embodiments, R₁ is C₁ alkyl. According to some embodiments, R₁ is methyl. According to some embodiments, R₂ is a linear unsubstituted C₁-C₁₅ alkyl. According to some embodiments, wherein R₂ is a linear unsubstituted C₅ alkyl.

According to some embodiments, the compound is represented by the structure of Formula (II),

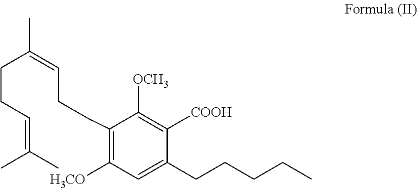 [SMILES: CCCCCc1cc(OC)c(CC/C=C(/C)CCC=C(C)C)c(OC)c1C(=O)O]

and stereoisomers, geometrical, and salts thereof.

According to some embodiments, the compound is represented by the structure of Formula (III) (designated herein EPM 310),

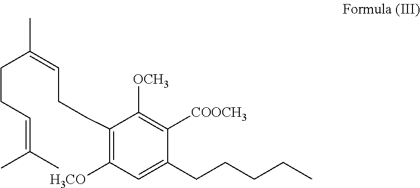 [SMILES: CCCCCc1cc(OC)c(CC/C=C(/C)CCC=C(C)C)c(OC)c1C(=O)OC]

and stereoisomers, geometrical, and salts thereof.

According to some embodiments, the pharmaceutical composition is administered in combination with at least one additional active pharmaceutical ingredient, wherein the at least one additional active pharmaceutical ingredient comprises at least one additional cannabinoid compound. According to some embodiments, the pharmaceutical composition comprises at least one additional active pharmaceutical ingredient, wherein the at least one additional active pharmaceutical ingredient comprises at least one additional cannabinoid compound.

According to some embodiments, the additional cannabinoid compound is selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), esters thereof and combination thereof.

According to some embodiments, the additional cannabinoid compound comprises a cannabigerolic acid (CBGA) ester represented by Formula (IV), a cannabinolic acid (CBNA) ester represented by Formula (V), a cannabidiolic acid (CBDA) ester represented by Formula (VI), a tetrahydrocannabinolic acid (THCA) ester represented by Formula (VII) or any combination thereof,

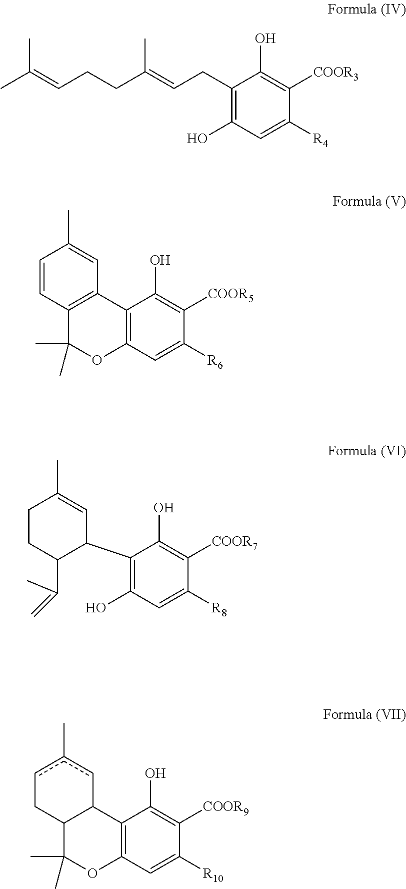

wherein
each of R₃, R₄, R₅, R₆, R₇, R₈, R₉ and R₁₀ is independently selected from the group consisting of a linear or branched, unsubstituted or substituted C₁-C₁₅ alkyl, a linear or branched, unsubstituted or substituted C₂-C₁₅ alkenyl, and a linear or branched, unsubstituted or substituted C₂-C₁₅ alkynyl;
one of the dashed lines in Formula (VII) represents a double bond and the other dashed line represents a single bond;
and stereoisomers, geometrical, and salts thereof.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides novel cannabigerolic acid (CBGA) derivatives including acids and esters, and uses thereof. The present invention further provides pharmaceutical compositions comprising CBGA derivatives, alone or in combination with one or more additional cannabinoid compounds, for use in treating various diseases and disorders and symptoms, including but not limited to pain, impaired neurological function, inflammation including respiratory inflammation, inflammatory bowel disease and autoimmune diseases, nausea, vomiting, convulsions, low appetite and glaucoma.

According to one aspect, the present invention provides a compound represented by the structure of Formula (I),

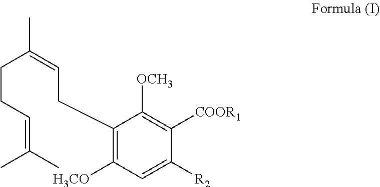

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, the compound is represented by the structure of Formula (II),

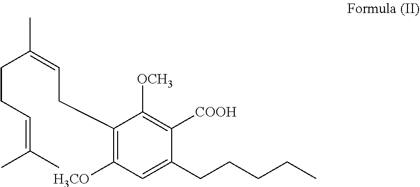 [SMILES: CCCCCc1cc(OC)c(C/C=C(/C)CCC=C(C)C)c(OC)c1C(=O)O]

and stereoisomers, geometrical, and salts thereof.

According to some embodiments, the compound is represented by the structure of Formula (III) (designated herein EPM 310),

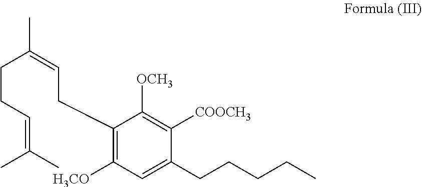 [SMILES: CCCCCc1cc(OC)c(C/C=C(/C)CCC=C(C)C)c(OC)c1C(=O)OC]

and stereoisomers, geometrical, and salts thereof.

According to some embodiments, R₁ is hydrogen. According to some embodiments, R₁ is a linear unsubstituted C₁-C₁₅ alkyl. According to some embodiments, R₁ is a linear unsubstituted C₂ alkyl. According to some embodiments, R₁ is a linear unsubstituted C₃ alkyl. According to some embodiments, R₁ is a linear unsubstituted C₁-C₄ alkyl. According to some embodiments, R₁ is a linear unsubstituted C₅ alkyl. According to some embodiments, R₁ is a linear substituted C₁-C₁₅ alkyl. According to some embodiments, R₁ is a branched unsubstituted C₁-C₁₅ alkyl. According to some embodiments, R₁ is a branched substituted C₁-C₁₅ alkyl. According to some embodiments, R₁ is methyl. According to some embodiments, R₁ is a linear unsubstituted C₂-C₁₅ alkenyl. According to some embodiments, R₁ is a linear substituted C₂-C₁₅ alkenyl. According to some embodiments, R₁ is a branched unsubstituted C₂-C₁₅ alkenyl. According to some embodiments, R₁ is a branched substituted C₂-C₁₅ alkenyl. According to some embodiments, R₁ is a linear unsubstituted C₂-C₁₅ alkynyl. According to some embodiments, R₁ is a linear substituted C₂-C₁₅ alkynyl. According to some embodiments, R₁ is a branched unsubstituted C₂-C₁₅ alkynyl. According to some embodiments, R₁ is a branched substituted C₂-C₁₅ alkynyl.

According to some embodiments, R₂ is a linear unsubstituted C₁-C₁₅ alkyl. According to some embodiments, R₂ is a linear substituted C₁-C₁₅ alkyl. According to some embodiments, R₂ is a branched unsubstituted C₁-C₁₅ alkyl. According to some embodiments, R₂ is a branched substituted C₁-C₁₅ alkyl. According to some embodiments, R₂ is a linear unsubstituted C₂ alkyl. According to some embodiments, R₂ is a linear unsubstituted C₃ alkyl. According to some embodiments, R₂ is a linear unsubstituted C₄ alkyl. According to some embodiments, R₂ is a linear unsubstituted C₅ alkyl. According to some embodiments, R₂ is a linear unsubstituted C₆ alkyl. According to some embodiments, R₂ is a linear unsubstituted C₂-C₁₅ alkenyl. According to some embodiments, R₂ is a linear substituted C₂-C₁₅ alkenyl. According to some embodiments, R₂ is a branched unsubstituted C₂-C₁₅ alkenyl. According to some embodiments, R₂ is a branched substituted C₂-C₁₅ alkenyl. According to some embodiments, R₂ is a linear unsubstituted C₂-C₁₅ alkynyl. According to some embodiments, R₂ is a linear substituted C₂-C₁₅ alkynyl. According to some embodiments, R₂ is a branched unsubstituted C₂-C₁₅ alkynyl. According to some embodiments, R₂ is a branched substituted C₂-C₁₅ alkynyl.

It is to be understood that the compounds provided herein may contain one or more chiral centers. Such chiral centers may each be of either of the (R) or (S) configuration. In case a compound of the invention contains more than one chiral center, each one of those chiral centers may be of the (R) or (S) configuration, independently. Thus, the compounds provided herein may be enantiomerically pure, or be stereoisomeric or diastereomeric mixtures.

The present invention intends to encompass all structural and geometrical isomers of a compound having a carbon-carbon double bond, including cis, trans, E and Z isomers, independently at each occurrence. It is apparent to a person of skill in the art that any of the structures drawn herein in one configuration (e.g., cis or trans, E or Z), also includes geometrical isomers thereof.

An “alkyl” group refers to a saturated aliphatic hydrocarbon, including straight-chain also referred to as linear-chain and branched-chain. In one embodiment, the alkyl group has 1-15 carbons designated here as C₁-C₁₅-alkyl. In another embodiment, the alkyl group has 2-6 carbons designated here as C₂-C₆-alkyl. In another embodiment, the alkyl group has 2-4 carbons designated here as C₂-C₄-alkyl. Each possibility represents a separate embodiment of the invention. The alkyl group may be unsubstituted or substituted by one or more groups selected from the group consisting of hydroxyl, halogen, amino, thiol, phosphate, and combinations thereof. Thus, the phrase “unsubstituted or substituted alkyl” refers to an alkyl group which is either unsubstituted (i.e. it is a hydrocarbon) or to an alkyl substituted with at least one of hydroxyl, halogen, amino, thiol, phosphate, and combinations thereof.

The terms “halo” and “halogen” refer to the fluoro, chloro, bromo or iodo atoms. There can be one or more halogens, which are the same or different.

An “alkenyl” group refers to an aliphatic hydrocarbon group containing at least one carbon-carbon double bond including straight-chain, linear-chain, branched-chain and cyclic alkenyl groups. In one embodiment, the alkenyl group has 2-15 carbon atoms (a C₂₋₁₅ alkenyl). In another embodiment, the alkenyl group has 2-4 carbon atoms in the chain (a C₂₋₄ alkenyl). Exemplary alkenyl groups include, but are not limited to, ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined herein for alkyl. Thus, the phrase “unsubstituted or substituted alkenyl” refers to an alkenyl group which is either unsubstituted (i.e. it is a hydrocarbon) or to an alkenyl substituted with at least one of hydroxyl, halogen, amino, thiol, phosphate, and combinations thereof.

An “alkynyl” group refers to an aliphatic hydrocarbon group containing at least one carbon-carbon triple bond including straight-chain or linear-chain and branched-chain. In one embodiment, the alkynyl group has 2-15 carbon atoms in the chain (a C₂₋₁₅ alkynyl). In another embodiment, the alkynyl group has 2-4 carbon atoms in the chain (a C₂₋₄ alkynyl). Exemplary alkynyl groups include, but are not limited to, ethynyl, propynyl, n-butynyl, 2-butynyl, 3-methylbutynyl, n-pentynyl, heptynyl, octynyl and decynyl. An alkylalkynyl is an alkyl group as defined herein bonded to an alkynyl group as defined herein. The alkynyl group can be unsubstituted or substituted through available carbon atoms with one or more groups defined herein for alkyl. Thus, the phrase “unsubstituted or substituted alkynyl” refers to an alkynyl group which is either unsubstituted (i.e. it is a hydrocarbon) or to an alkynyl substituted with at least one of hydroxyl, halogen, amino, thiol, phosphate, and combinations thereof.

According to another aspect, the present invention provides a pharmaceutical composition comprising a therapeutically effective amount of a compound represented by the structure of Formula (I) and a pharmaceutically acceptable carrier or excipient,

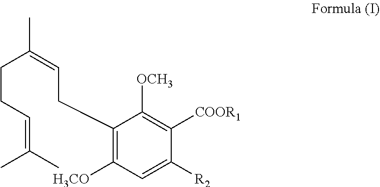

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

The word “cannabinoid” as used herein refers to any compound that interacts with cannabinoid receptors including endocannabinoids (produced naturally in the body by humans and animals), phytocannabinoids (found in cannabis and some other plants), and synthetic cannabinoids (manufactured artificially). The term “cannabinoid acid” refers to the acid form of the above-mentioned cannabinoids.

The word “cannabigerol” refers to cannabigerol (CBG) and CBG derivatives. CBG may be obtained from industrial hemp extract with a trace amount of THC and/or CBD or from cannabis extract using high CBG cannabis cultivars. According to some embodiments, cannabigerol may be obtained from plant extract, or may be prepared synthetically (manufactured artificially), the structure of CBG is presented below:

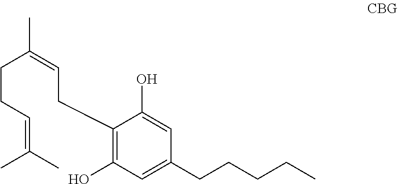 [SMILES: CCCCCc1cc(O)c(C/C=C(/C)CCC=C(C)C)c(O)c1]

The abbreviation “CBGA” is used herein to refer to the common cannabigerolic acid, which is the acid form of CBG. The term “cannabigeolic acid ester” or “cannabigerolic ester” refers to various molecules, which are the alkyl, alkenyl, or alkynyl form of CBGA. The structure of CBGA is presented below:

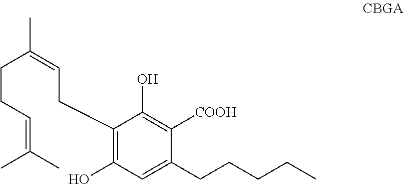 [SMILES: CCCCCc1cc(O)c(C/C=C(/C)CCC=C(C)C)c(O)c1C(=O)O]

The term “cannabigerolic acid derivative” refers to derivatives of CBGA, which are substituted at positions 1, 2 and 5 of the aromatic ring by a methoxy group at positions 1 and 5 and by a carboxylic acid at position 2. The term “cannabigerolic acid ester derivative” refers to various molecules, which are the alkyl, alkenyl or alkynyl form of CBGA derivative as described herein above. The chemical structures of CBGA and CBGA ester derivatives are presented below:

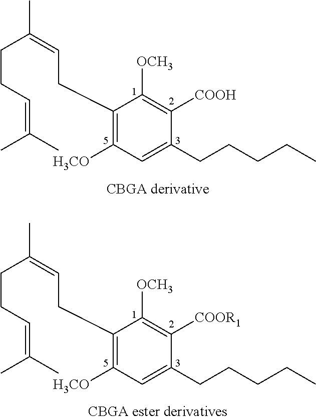

wherein
R₁ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;

It is to be understood that although the common cannabigerolic acid (CBGA) isomers include an n-C₅H₁₁ chain at position 3, derivatives of CBGA may include other substituents, in particular alkyl, alkenyl or alkynyl groups. Therefore, the term cannabigerolic acid/ester derivative includes corresponding structures, in which position 3 is substituted by a group, which is either an n-C₅H₁₁ or a different chemical group, in particular alkyl, alkenyl or alkynyl groups. The term “cannabigerolic acid” and/or cannabigerolic acid ester” should be interpreted broadly referring to all possible stereoconfigurations, geometrical, and salts of the relevant formula.

The present invention thus provides pharmaceutical compositions comprising the compounds disclosed herein and a pharmaceutically acceptable carrier and/or excipient. The compounds of the present invention can be safely administered orally or non-orally. Routes of administration include, but are not limited to, oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual. Each possibility represents a separate embodiment. The pharmaceutical compositions can be formulated as tablets (including e.g. film-coated tablets), powders, granules, capsules (including soft capsules), orally disintegrating tablets, and sustained-release preparations as is well known in the art. Each possibility represents a separate embodiment.

According to some embodiments, the pharmaceutical composition is formulated into a dosage form suitable for, oral, intranasal, intravenous, intraarterial, topical, intramuscular, transdermal, intraperitoneal, intrathecal, vaginal, rectal or subcutaneous administration.

According to some embodiments, the pharmaceutical composition is formulated as tablet, pill, capsule (e.g. soft or hard gelatin capsule), pellets, granules, powder, a wafer, coated or uncoated beads, lozenge, sachet, cachet, elixir, a depot system, a patch, suspension, dispersion, emulsion, solution, syrup, aerosol, oil, ointment, suppository, a gel, and a cream. Each possibility represents a separate embodiment.

For preparing solid compositions such as tablets, the active pharmaceutical ingredient is mixed with a pharmaceutical carrier or excipient to form a solid pre-formulation composition containing a substantially homogeneous distribution of the compound disclosed herein in the pharmaceutical carrier or excipient.

Any method can be used to prepare the pharmaceutical compositions. For example, solid dosage forms can be prepared by wet granulation, dry granulation, direct compression and the like as is known in the art. The liquid forms in which the compositions disclosed herein may be incorporated, for administration via a route selected from oral, topical or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Each possibility represents a separate embodiment.

According to some embodiments, the composition may be formulated as single-phase aqueous, emulsion or multiple emulsions. According to some embodiments, the composition is formulated as emulsion. These emulsions may be oil-in-water (o/w) emulsions, water-in-oil (w/o) emulsions, or multiple emulsions such as oil-in-water-in-oil (o/w/o) or water-in-oil-in-water (w/o/w). It is understood that the oil phase can comprise silicone oils, non-silicone organic oils, or mixtures thereof. The compositions can comprise two immiscible phases that are reconstituted prior to use. Each possibility represents a separate embodiment.

Compositions for inhalation include solutions and suspensions in pharmaceutically acceptable aqueous or organic solvents, or mixtures thereof, and powders. The liquid or solid compositions may contain suitable pharmaceutically acceptable carriers or excipients as described herein. The compositions may be administered by the oral or nasal respiratory route. Compositions may also be nebulized by use of inert gases. Nebulized solutions may be breathed directly from the nebulizing device or the nebulizing device may be attached to a face mask tent, or intermittent positive pressure breathing machine. Solution, suspension, or powder compositions may be administered, inter alia, orally or nasally, from devices that deliver the formulation in an appropriate manner.

According to some embodiments, the composition is prepared for topical administration, e.g. as an oil, ointment, gel or cream. Adjuvants for topical administration may include, for example, sodium carboxymethylcellulose, polyacrylates, polyoxyethylene-polyoxypropylene-block polymers, polyethylene glycol and wood wax alcohols. The term “gel” as used herein, refers to a substantially dilute cross-linked system, which exhibits little or no flow when in the steady-state having a solid jelly-like matrix. As contemplated herein, gel may comprise hydrogel, organogel, thermosensitive gel, non-thermosensitive gel, and aerogel. Each possibility represents a separate embodiment.

Hydrogels are highly hydrated, macromolecular networks, dispersed in water or other biological fluids. Hydrogels that exhibit the specific property of increased viscosity with increased temperatures are known as thermosensitive (or thermosetting) hydrogels. Such hydrogels have been shown to have easier application combined with longer survival periods at the site of application as compared to non-thermosensitive hydrogels, and are therefore advantageous as sustained-release drug delivery systems. Gel may be characterized by a critical gelation temperature, wherein gelation is effected at the critical gelation temperature or at temperatures above the critical gelation temperature. As used herein, “critical gelation temperature” refers to the lowest temperature at which some gelation of a material is observed (e.g., by increase in shear storage modulus).

According to some embodiments, the pharmaceutical composition can be formulated for local administration into the vagina. In accordance with these embodiments, the compositions may also be embedded in an article or administered using an applicator. Suitable forms for vaginal administration include, but are not limited to, bandage, suppository, pessary, douche, ointment, tablet, tampon, foam, cream, gel, paste, microcapsules, vaginal sponge, vaginal ring, and syringe-like applicator. Each possibility represents a separate embodiment. An optional carrier for vaginal administration comprises a lipid carrier, e.g. fatty acids, glycerin and pharmaceutically acceptable oils.

The pharmaceutical compositions disclosed herein may exhibit release mode which may be immediate release, controlled release or a mixture thereof. Each possibility represents a separate embodiment. “Immediate release” (IR) compositions in the context of the present invention refers to compositions in which the active ingredient is released without delay following administration. “Controlled release” (CR) compositions in the context of the present invention refers to compositions in which the active ingredient is released gradually over a period of time following administration.

Suitable pharmaceutically acceptable carriers or excipients include, but are not limited to, a binder, a filler, a diluent, a surfactant or emulsifier, a glidant or lubricant, buffering or pH adjusting agent, a tonicity enhancing agent, a wetting agent, a preservative, an antioxidant, a flavoring agent, a colorant, and a mixture or combination thereof. Each possibility represents a separate embodiment.

Suitable binders include, but are not limited to, polyvinylpyrrolidone, copovidone, hydroxypropyl methylcellulose, starch, and gelatin. Each possibility represents a separate embodiment.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose. Each possibility represents a separate embodiment.

Suitable lubricants include, but are not limited to, sodium stearyl fumarate, stearic acid, polyethylene glycol or stearates, such as magnesium stearate. Each possibility represents a separate embodiment.

Suitable diluents include, but are not limited to, dicalcium phosphate dihydrate, sugars, lactose, calcium phosphate, cellulose, kaolin, mannitol, sodium chloride, and dry starch. Each possibility represents a separate embodiment.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil. Each possibility represents a separate embodiment.

Suitable glidants or lubricants include, but are not limited to, colloidal silicon dioxide, magnesium stearate, talc, and mineral oil. Each possibility represents a separate embodiment.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide. Each possibility represents a separate embodiment.

Suitable tonicity enhancing agents include, but are not limited to, ionic and non-ionic agents such as, alkali metal or alkaline earth metal halides, urea, glycerol, sorbitol, mannitol, propylene glycol, and dextrose. Each possibility represents a separate embodiment.

Suitable wetting agents include, but are not limited to, glycerin, cetyl alcohol, and glycerol monostearate. Each possibility represents a separate embodiment.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide. Each possibility represents a separate embodiment.

Suitable antioxidants include, but are not limited to, sorbic acid, ascorbic acid, ascorbate, glycine, α-tocopherol, butylated hydroxyanisole (BHA), and butylated hydroxytoluene (BHT). Each possibility represents a separate embodiment.

Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot. Each possibility represents a separate embodiment.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide. Each possibility represents a separate embodiment.

It is contemplated that the compounds include any solvate thereof. The term “solvate” as used herein refers to a physical association of a compound disclosed herein with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates and the like. A “hydrate” is a solvate in which the solvent molecule is water.

In embodiments in which the compound is incorporated into a pharmaceutical composition in solid state form, the present disclosure also includes any polymorphs thereof. The term “polymorph” refers to a particular crystalline or amorphous state of a substance, which can be characterized by particular physical properties such as X-ray diffraction, electron diffraction, IR spectra, Raman spectra, melting point, and the like.

Any of the cannabinoids disclosed herein, specifically, the abnormal cannabidiolic acids and their esters, can be prepared synthetically or semi-synthetically.

Natural cannabinoids may be isolated or extracted from a natural source or prepared by synthetic or semi-synthetic means. For example, cannabinoids can be isolated by extraction from cannabis plants. Plants in the Cannabis genus include, but are not limited to, Cannabis sativa, Cannabis ruderalis, and Cannabis indica. Each possibility represents a separate embodiment. These plants are the natural sources of cannabinoids.

According to some embodiments, the pharmaceutical composition further comprising at least one additional active pharmaceutical ingredient.

According to some embodiments, the additional active pharmaceutical ingredient comprises at least one additional cannabinoid compound.

According to some embodiments, the additional cannabinoid compound is selected from the group consisting of cannabidiol (CBD), cannabigerol (CBG), Δ⁸-tetrahydrocannabinol (Δ⁸-THC), Δ⁹-tetrahydrocannabinol (Δ⁹-THC), cannabinol (CBN), Δ⁹(11)-tetrahydrocannabinol (exo-THC), cannabichromene (CBC), tetrahydrocannabinol-C3 (THC-C3), tetrahydrocannabinol-C4 (THC-C4), tetrahydrocannabinol-C7 (THC-C7), esters thereof and combinations thereof. Each possibility represents a separate embodiment of the present invention.

According to some embodiments, the additional cannabinoid compound comprises a cannabigerolic acid (CBGA) ester represented by Formula (IV), a cannabinolic acid (CBNA) ester represented by Formula (V), a cannabidiolic acid (CBDA) ester represented by Formula (VI), a tetrahydrocannabinolic acid (THCA) ester represented by Formula (VII) or any combination thereof,

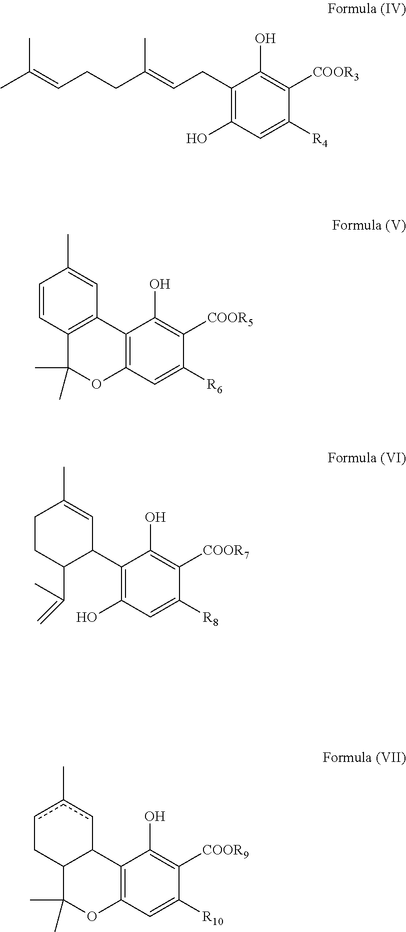

wherein
each of R₃, R₄, R₅, R₆, R₇, R₈, R₉ and R₁₀ is independently selected from the group consisting of a linear or branched, unsubstituted or substituted C₁-C₁₅ alkyl, a linear or branched, unsubstituted or substituted C₂-C₁₅ alkenyl, and a linear or branched, unsubstituted or substituted C₂-C₁₅ alkynyl;
one of the dashed lines in Formula (VII) represents a double bond and the other dashed line represents a single bond;
and stereoisomers, geometrical, and salts thereof.

According to some embodiments, the additional cannabinoid compound is cannabidiolic acid methyl ester.

According to some embodiments, the additional cannabinoid compound is present in one or more extracts of a cannabis plant. According to some embodiments, the cannabis plant extract is obtained from a strain selected from the group consisting of Cannabis sativa, Cannabis indica, Cannabis ruderalis, a hybrid strain, and combinations thereof. According to some embodiments, the cannabis plant extract is obtained from a strain selected from the group consisting of a high-CBD strain, a high-THC strain, and a combination thereof.

According to some embodiments, the cannabis plant extract comprises at least one cannabinoid selected from the group consisting of cannabidiol (CBD), tetrahydrocannabinol (THC), cannabinol (CBN), cannabigerol (CBG), cannabichromene (CBC), acids thereof and combinations thereof.

According to some embodiments, the cannabis plant extract comprises from about 1 to about 25% (w/w) of CBD. According to some embodiments, the cannabis plant extract comprises about 1% (w/w) of CBD. According to some embodiments, the cannabis plant extract comprises about 10% (w/w) of CBD. According to some embodiments, the cannabis plant extract comprises about 25% (w/w) of CBD.

According to some embodiments, the cannabis plant extract comprises from about 1 to about 25% (w/w) of THC. According to some embodiments, the cannabis plant extract comprises about 1% (w/w) of THC. According to some embodiments, the cannabis plant extract comprises about 10% (w/w) of THC. According to some embodiments, the cannabis plant extract comprises about 25% (w/w) of THC.

The term “extract” as used herein refers a product prepared by extraction by physical means (e.g. by comminuting, pressing, heating, pulsed electric field assisted treatments, shear treatments and/or pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence which is free of, or substantially free of solvent. The term extract may be a single extract obtained from a particular extraction step or series of extraction steps. Extract also may be a combination of extracts obtained from separate extraction steps or separate feedstocks. Such combined extracts are thus also encompassed by the term “extract”. Any methods of extraction with suitable solvent are encompassed. Exemplary extraction methods can be found for example in U.S. Pat. No. 6,403,126, the contents of which are incorporated by reference herein. The extract may be obtained from any part of the plant e.g. from leaves, flowers, stems, roots, fruits and seeds. The extract may be aqueous or oily.

According to some embodiments, the cannabis plant extract is formed through contact with a suitable solvent or a combination of solvents. According to some embodiments, the solvent is selected from the group consisting of a polar solvent, a hydrocarbon solvent, carbon dioxide, and a combination thereof.

The term “extract” further refers to a liquid or semi-solid or resinous substance obtained through extraction from plants defined in the present application, i.e. extracts obtained from cannabis plant e.g. Cannabis sativa, Cannabis indica, and Cannabis ruderalis. In some embodiments, the term refers to a mixture of liquid or semi-solid, resinous substances obtained through extraction from two or more different plants. In some embodiments, the term refers also to a compound purified from the extract. According to some embodiments, the term “extract” has the meaning of a mixture or combination of two or more extracts.

The term “cannabis extract” as used herein refers to one or more plant extracts from the cannabis plant. A cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to supercritical or subcritical extraction with CO₂, extraction with hot or cold gas and extraction with solvents. In some embodiments, the term refers to a mixture of liquid or semi-solid, resinous substances obtained through extraction from two or more different cannabis species. In some embodiments, the term refers also to a compound purified from the extract. The term “cannabis plant” as used herein, refers to plants of the genus Cannabis, including but not limited to Cannabis sativa, Cannabis indica, and Cannabis ruderalis. According to some embodiments, cannabis plant is a CBD-rich strain of cannabis plant or THC-rich strain of cannabis plant. Each possibility represents a separate embodiment.

The term “hybrid strain” refers to different strains of Cannabis which include differing amounts and/or ratios of the various cannabinoid compounds. For example, Cannabis sativa typically has a relatively high THC/CBD ratio. Conversely, Cannabis indica has a relative low THC/CBD ratio compared to Cannabis sativa (although the absolute amount of THC can be higher in Cannabis indica than in Cannabis sativa).

As used herein the terms “high-CBD strain” and “CBD-rich strain” refer to a strain of cannabis plant which comprises CBD and optionally one or more additional cannabinoids, such as, for example, but not limited to: THC, CBN, and the like. According to some embodiments, CBD is the main component in the high-CBD strain.

As used herein the terms “high-THC strain” and “THC-rich strain” are directed to a strain of cannabis plant which comprises THC and optionally one or more additional cannabinoids, such as, for example, but not limited to: CBD, CBN, and the like. According to some embodiments, THC is the main component in the high-THC strain.

According to another aspect, the present invention provides a pharmaceutical composition comprising a therapeutically effective amount of a compound represented by the structure of Formula (I) and a pharmaceutically acceptable carrier or excipient, for use in the treatment of a disease, disorder or symptom amenable to treatment with CBG,

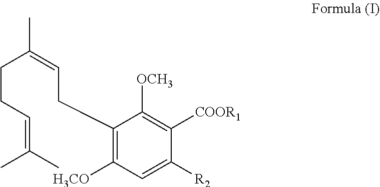

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, the disease, disorder or symptom amenable to treatment with cannabigerol (CBG) is selected from the group consisting of pain, autoimmune disease, cancer, bacterial infection, impaired neurological function, inflammation, nausea, vomiting, convulsions, low appetite and glaucoma.

According to some embodiments, the composition disclosed herein is for use in the treatment of pain. According to some embodiments, the composition disclosed herein is for use in the attenuation of pain. According to some embodiments, the composition disclosed herein is for use in the alleviation of pain.

According to some embodiments, the composition disclosed herein is for use in the treatment of autoimmune disease.

According to some embodiments, the composition disclosed herein is for use in the treatment of cancer. According to some embodiments, the composition disclosed herein is for use as an anti-tumor agent.

According to some embodiments, the composition disclosed herein is for use in the treatment of bacterial infection. According to some embodiments, the composition disclosed herein is for use as an anti-bacterial agent.

According to some embodiments, the composition disclosed herein is for use in the treatment of impaired neurological function.

According to some embodiments, the composition disclosed herein is for use in the treatment of impaired neurological function selected from the group consisting of stroke, trauma, Parkinson's disease, vascular dementia, senile dementia, Alzheimer's disease, mild cognitive impairment, Huntington's Disease, Amyotrophic lateral sclerosis (ALS), epilepsy, multiple sclerosis, and psychiatric disorders. Each possibility represents a separate embodiment of the present invention.

According to some embodiments, the impaired neurological function is epilepsy.

According to some embodiments, the composition disclosed herein is for use in the treatment of psychiatric disorders. According to some embodiments, the psychiatric disorder is selected from the group consisting of depression, anxiety, acute or chronic stress, schizophrenia, panic attacks, ADHD, bipolar disorder, obsessive compulsive disorder or personality disorders.

According to some embodiments, the composition disclosed herein is for use in the treatment of depression. According to some embodiments, the composition disclosed herein is for use in the treatment of anxiety. According to some embodiments, the composition disclosed herein is for use in the treatment of acute or chronic stress. According to some embodiments, the composition disclosed herein is for use in the treatment of schizophrenia. According to some embodiments, the composition disclosed herein is for use in the treatment of panic attacks. According to some embodiments, the composition disclosed herein is for use in the treatment of ADHD. According to some embodiments, the composition disclosed herein is for use in the treatment of bipolar disorder. According to some embodiments, the composition disclosed herein is for use in the treatment of obsessive-compulsive disorder.

According to some embodiments, the composition disclosed herein is for use in the treatment of inflammation.

According to some embodiments, the composition disclosed herein is for use in treating joint inflammatory diseases and joint degeneration. According to additional embodiments, the pharmaceutical composition is for use in treating respiratory inflammation. According to additional embodiments, the composition disclosed herein is for use in treating inflammatory bowel disease.

According to some embodiments, the inflammation is respiratory inflammation.

According to some embodiments, the respiratory inflammation is acute respiratory inflammation.

The respiratory inflammation can be associated with a disease or disorder including, but not limited to, asthma, chronic obstructive airway disorder, chronic obstructive pulmonary disease (COPD), pneumonia, respiratory syncytial viral infection, bronchitis, bronchiolitis, idiopathic pulmonary fibrosis, cystic fibrosis, acute respiratory distress syndrome (ARDS), bronchopulmonary dysplasia, occupational respiratory disease, particulate exposure, pleurisy, emphysema, and pulmonary edema.

According to some embodiments, the respiratory inflammation is chronic respiratory inflammation. According to certain embodiments, the respiratory inflammation is pneumonitis. According to certain embodiments, the respiratory inflammation is asthma. According to certain embodiments, the respiratory inflammation is COPD.

According to some embodiments, the respiratory inflammation is caused by a pathogen. According to certain embodiments, the respiratory inflammation is caused by a pathogen selected from the group consisting of bacteria, viruses, parasites and fungi.

According to some embodiments, the respiratory inflammation is caused by a virus. According to additional embodiments, the respiratory inflammation is caused by Severe acute respiratory syndrome (SARS) virus. According to additional embodiments, the respiratory inflammation is caused by SARS-CoV-2 (Covid-19).

According to some embodiments, the composition disclosed herein is for use as a neuroprotective agent. According to some embodiments, the composition disclosed herein is for use in treating multiple sclerosis.

According to some embodiments, the composition disclosed herein is for use in treating cancer.

According to some embodiments, the composition disclosed herein is for use in the treatment of a symptom selected from the group consisting of nausea, vomiting and low appetite. According to some embodiments, the composition disclosed herein is for use in the treatment of nausea. According to some embodiments, the composition disclosed herein is for use in the treatment of vomiting. According to some embodiments, the composition disclosed herein is for use in the treatment of low appetite.

According to some embodiments, the composition disclosed herein is for use in the treatment of convulsions.

According to some embodiments, the composition disclosed herein is for use in the treatment of glaucoma.

According to some embodiments, the composition disclosed herein is for use in the treatment of gastrointestinal diseases or disorders.

According to some embodiments, the composition disclosed herein is for use in the treatment of Non-Alcoholic Fatty Liver Disease (NAFLD), chronic kidney disease (CKD), obesity, hyperglycemia, diabetes, metabolic syndrome and/or obesity related diseases.

According to some embodiments, the composition disclosed herein is for use in the treatment of obesity.

According to some embodiments, the composition disclosed herein is for use in the treatment of muscular dystrophy.

According to another aspect, the present invention provides a method of treating a disease, disorder or symptom amenable to treatment with cannabigerol (CBG), the method comprising administering to a subject in need thereof a pharmaceutical composition comprising a therapeutically effective amount of a compound represented by the structure of Formula (I) and a pharmaceutically acceptable carrier or excipient,

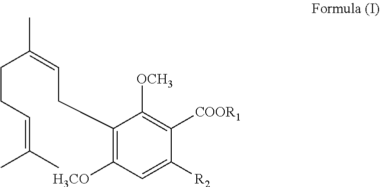

wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical, and salts thereof.

According to some embodiments, the method of treating, a disease, disorder or symptom is selected from the group consisting of impaired neurological function, inflammation, nausea, vomiting, convulsions, low appetite, and glaucoma, the method comprising administering to a subject in need thereof the pharmaceutical composition disclosed herein.

According to some embodiments, the impaired neurological function is selected from the group consisting of stroke, trauma, Parkinson's disease, vascular dementia, senile dementia, Alzheimer's disease, mild cognitive impairment, Huntington's Disease, Amyotrophic lateral sclerosis (ALS), epilepsy, multiple sclerosis, and psychiatric disorders.

According to some embodiments, there is provided a method of treating, attenuating or alleviating pain, the method comprising administering to a subject in need thereof the pharmaceutical composition disclosed herein.

According to some embodiments, there is provided a method of treating, attenuating or alleviating pain, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, the pharmaceutical composition is administered in combination with at least one additional active pharmaceutical ingredient as described herein.

Other suitable additional active pharmaceutical ingredients include agents for pain relief such as non-steroidal anti-inflammatory agents (NSAIDs). Suitable NSAIDs include, but are not limited to, acetyl salicylic acid, indometacin, sulindac, phenylbutazone, diclofenac, fentiazac, ketorolac, piroxicam, tenoxicam, mecoxicam, meloxicam, cinnoxicam, ibufenac, ibuprofen, naproxen, ketoprofen, nabumetone, niflumic acid, nimesulide, and pharmaceutically acceptable salts thereof. Each possibility represents a separate embodiment of the invention.

A group of NSAIDs within the scope of the present invention are Cox-2 inhibitors. Cox-2 inhibitors suitable for use in the combinations of the present invention include, but are not limited to, (4-(4-cyclohexyl-2-methyloxazol-5-yl)-2-fluorobenzenesulfonamide), 5-chloro-3-(4-(methylsulfonyl)phenyl)-2-(methyl-5-pyridinyl)pyridine, 2-(3,5-difluorophenyl)-3-4 (methylsulfonyl)phenyl)-2-cyclopenten-1-one, 4-[5-(4-methylphenyl)-3-(trifluoromethyl)-1H-pyrazol-1-yl]-benzenesulfonamide, rofecoxib, (4-(4-methylsulfonyl)phenyl]-3-phenyl-2(5H)-furanone), 4-(5-methyl-3-phenylisoxazol-4-yl)benzenesulfonamide, N-[[4-(5-methyl-3-phenylisoxazol-4yl]phenyl]sulfonyl]propanamide, 4-[5-(4-chlorophenyl)-3-(trifluoromethyl)-1H-pyrazole-1-yl]benzenesulfonamide, N-(2,3-dihydro-1,1-dioxido-6-phenoxy-1,2-benzisothiazol-5-yl) methanesulfonamide, 6-[[5-(4-chlorobenzoyl)-1,4-dimethyl-1H-pyrrol-2-yl]methyl]-3(2H)-pyridazinone, N-(4-nitro-2-phenoxyphenyl) methanesulfonamide, 3-(3,4-difluorophenoxy)-5,5-dimethyl-4-[4-(methylsulfonyl)phenyl]-2 (SH)-furanone, N-[6-[(2,4-difluorophenyl)thio]-2,3-dihydro-1-oxo-1H-inden-5-yl]methanesulfonamide, 3-(4-chlorophenyl)-4-[-4-(methylsulfonyl)phenyl]-2 (3H)-oxazolone, 4-[3-(4-fluorophenyl)-2,3-dihydro-2-oxo-4-oxazolyl]benzenesulfonamide, 3-[4-(methylsulfonyl)phenyl]-2-phenyl-2-cyclopenten-1-one, 4-(2-methyl-4-phenyl-5-oxazolyl)benzenesulfonamide, 3-(4-fluorophenyl)-4-[4-(methylsulfonyl)phenyl]-2 (3H)-oxazolone, 5-(4-fluorophenyl)-1-[4-(methylsulfonyl)phenyl]-3-(trifluoromethyl)-1H-pyrazole, 4-[5-phenyl)-3-(trifluoromethyl)-1H-pyrazol-1-yl)benzenesulfonamide, 4-[1-phenyl-3-(trifluoromethyl)-1H-pyrazol-5-yl]benzenesulfonamide, 4-[5-(4-fluorophenyl)-3-(trifluoromethyl)-1H-pyrazol-1-yl]benzenesulfonamide, N-[2-(cyclohexyloxy)-4-nitrophenyl]methanesulfonamide, N-[6-(2,4-difluorophenoxy)-2,3-dihydro-1-oxo-1H-inden-5-yl]methanesulfonamide, 3-(4-chlorophenoxy-4-[(methylsulfonyl)amino]benzenesulfonamide, 3-(4-chlorophenoxy-4-[(methylsulfonyl)amino]benzenesulfonamide, 3-[(1-methyl-1H-imidzaol-2-yl)thio]-4 [(methylsulfonyl)amino]benzenesulfonamide, 5,5-dimethyl-4-[4-(methylsulfonyl)phenyl]-3-phenoxy-2(5H)-furanone, N-[6-[(4-ethyl-2-thiazolyl)thio]-1,3-dihydro-1-oxo-5-isobenzofuranyl]methanesulfonamide, 3-[(2,4-dichlorophenyl)thio]-4-[(methylsulfonyl)amino]benzenesulfonamide, 1-fluoro-4-[2-[4-(methylsulfonyl)phenyl]cyclopenten-1-yl]benzene, 4-[5-(4-chlorophenyl)-3-(difluoromethyl)-1H-pyrazol-1-yl]benzenesulfonamide, 3-[1-[4-(methylsulfonyl)phenyl]-4-(trifluoromethyl)-1H-imidazol-2-yl]pyridine, 4-[2-(3-pyridinyl)-4-(trifluoromethyl)-1H-imidazol-1-yl]benzenesulfonamide, 4-[5-(hydroxymethyl)-3-phenylisoxazol-4-yl]benzenesulfonamide, 4-[3-(4-chlorophenyl)-2,3-dihydro-2-oxo-4-oxazolyl]benzenesulfonamide, 4-[5-(difluoromethyl)-3-phenylisoxazol-4-yl]benzenesulfonamide, [1,1′:2′,1″-terphenyl]-4-sulfonamide, 4-(methylsulfonyl)-1,1′,2],1″-terpheynyl, 4-(2-phenyl-3-pyridinyl)benzenesulfonamide, N-[3-(formylamino)-4-oxo-6-phenoxy-4H-1-benzopyran-7-yl]methanesulfonamide, darbufelone, celecoxib, rofecoxib, parecoxib, and valdecoxib. Each possibility represents a separate embodiment of the invention. Exemplary Cox-2 inhibitors include, but are not limited to, celecoxib, rofecoxib, parecoxib and valdecoxib. Each possibility represents a separate embodiment of the invention.

According to some embodiments, there is provided a method of treating psychiatric disorders, the method comprising administering to a subject in need thereof the pharmaceutical composition disclosed herein.

According to some embodiments, there is provided a method of treating psychiatric disorders, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, the pharmaceutical composition is administered in combination with at least one additional active pharmaceutical ingredient as described herein.

According to some embodiments, there is provided a method of treating impaired neurological function, the method comprising administering to a subject in need thereof the pharmaceutical composition disclosed herein.

According to some embodiments, there is provided a method of treating epilepsy comprising administering to a subject in need thereof the pharmaceutical composition disclosed herein.

According to some embodiments, there is provided a method of treating impaired neurological function, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, there is provided a method of treating epilepsy, the method comprising administering to a subject in need thereof a pharmaceutical composition comprising a therapeutically effective amount of a compound ester represented by the structure of Formula (I) as described herein, and a pharmaceutically acceptable carrier or excipient.

According to some embodiments, the pharmaceutical composition is administered in combination with at least one additional active pharmaceutical ingredient as described herein.

According to some embodiments, there is provided a method of treating inflammation, the method comprising administering to a subject in need thereof the pharmaceutical composition disclosed herein.

According to some embodiments, there is provided a method of treating inflammation, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, there is provided a method of treating joint inflammatory diseases and joint degeneration, the method comprising administering to a subject in need thereof a pharmaceutical composition comprising a therapeutically effective amount of a compound represented by the structure of Formula (I) as described herein above, and a pharmaceutically acceptable carrier or excipient.

According to some embodiments, there is provided a method of treating respiratory inflammation, the method comprising administering to a subject in need thereof a pharmaceutical composition comprising a therapeutically effective amount of a compound represented by the structure of Formula (I) as described herein above, and a pharmaceutically acceptable carrier or excipient.

According to some embodiments, there is provided a method of treating inflammatory bowel disease, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, the pharmaceutical composition is administered in combination with at least one additional active pharmaceutical ingredient as described herein.

According to some embodiments, there is provided a method of treating a symptom selected from the group consisting of nausea, vomiting and low appetite, the method comprising administering to a subject in need thereof the pharmaceutical composition disclosed herein. Each possibility represents a separate embodiment of the present invention.

According to some embodiments, there is provided a method of treating a symptom selected from the group consisting of nausea, vomiting and low appetite, the method comprising administering to a subject in need thereof a pharmaceutical composition comprising a therapeutically effective amount of a compound represented by the structure of Formula (I) as described herein, and a pharmaceutically acceptable carrier or excipient. Each possibility represents a separate embodiment of the present invention.

According to some embodiments, the pharmaceutical composition is administered in combination with at least one additional active pharmaceutical ingredient as described herein. According to some embodiments, the symptom is nausea. According to some embodiments, the symptom is vomiting. According to some embodiments, the symptom is low appetite.

According to some embodiments, there is provided a method of treating convulsions, the method comprising administering to a subject in need thereof the pharmaceutical composition disclosed herein.

According to some embodiments, there is provided a method of treating convulsions, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, the pharmaceutical composition is administered in combination with at least one additional active pharmaceutical ingredient as described herein.

According to some embodiments, there is provided a method of treating glaucoma, the method comprising administering to a subject in need thereof the pharmaceutical composition disclosed herein.

According to some embodiments, there is provided a method of treating glaucoma, the method comprising administering to a subject in need thereof a pharmaceutical composition comprising a therapeutically effective amount of a compound represented by the structure of Formula (I) as described herein, and a pharmaceutically acceptable carrier or excipient.

According to some embodiments, the pharmaceutical composition is administered in combination with at least one additional active pharmaceutical ingredient as described herein.

Although the pharmaceutical composition comprising the compound of the present invention may be administered as the single therapeutic agent for the treatment of diseases or disorders described herein, combination therapy including co-administration of one or more additional active pharmaceutical ingredients is within the scope of the present invention. Co-administration of the compound of the present invention with one or more therapeutic agents may take place sequentially in any order, simultaneously or a combination thereof. For example, administration of the compound of the present invention can take place prior to, after or at the same time as the administration of the additional therapeutic agent(s). For example, a total treatment period can be decided for the compound of the present invention. The additional agent(s) can be administered prior to the onset of treatment with the compound of the present invention or following treatment with the compound of the present invention. In addition, the additional agent(s) can be administered during the period of administering the compound of the present invention but does not need to occur over the entire treatment period. In another embodiment, the treatment regimen includes pre-treatment with one agent, followed by the addition of the other agent or agents. Alternating sequences of administration are also contemplated. Alternating administration includes administration of the compound of the present invention, followed by the additional agent, followed by the compound of the present invention, etc. The aforementioned sequences can also be administrated in several cycles wherein each cycle may be similar or different with each possibility representing a separate embodiment. The therapeutic efficacy of the combination of the compound of the present invention and the additional agent(s) is at least additive. In some embodiments, the therapeutic efficacy is synergistic, namely the overall dose of each of the components may be lower, thus resulting in significantly lower side effects experienced by the subject, while a sufficient desirable therapeutic effect is nonetheless achieved. When combination therapy is involved, the compound of the present invention and the additional therapeutic agent(s) may be provided in a single dosage form such as a fixed-dose combination or in separate compositions intended for simultaneous administration.

The precise dose to be employed in the pharmaceutical composition disclosed herein will depend on the route of administration, and the seriousness of the disease, and should be decided according to the judgment of the practitioner and each patient's circumstances. An optional dosage will be within the range of about 0.01-1000 mg/kg of body weight, about 0.1 mg/kg to 100 mg/kg, about 1 mg/kg to 100 mg/kg, about 10 mg/kg to 75 mg/kg, about 0.1 to 1 mg/kg etc., including each value within the specified range.

According to some embodiments, the composition comprises the compound of the present invention at a concentration of between 10 to 200 mg/ml.

The administration schedule will depend on several factors such as the severity and progression of the disease, disorder or symptom. For example, the compositions of the invention can be taken once-daily, twice-daily, thrice daily, once-weekly or once-monthly. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g. daily administration for two to eight consecutive weeks, then a rest period with no administration for up to one week) or it may mean administration on alternate days. The effectiveness of said compositions could enable a shortened period of treatment with superior results.

“A therapeutically effective amount” as used herein refers to an amount of an agent which is effective, upon single or multiple dose administration to the subject in providing a therapeutic benefit to the subject. In additional embodiments, the compounds of the present invention are used for the preparation of a medicament for treating any one of the diseases, disorders, conditions or symptoms disclosed herein.

The following examples describe specific aspects of the invention to illustrate the invention and provide a description of the present methods for those skilled in the art. It should be noted that the term “and” or the term “or” is generally employed in its sense including “and/or” unless the context clearly dictates otherwise. As used herein, the term “about” is meant to encompass variations of ±10%.

### EXAMPLES

### Synthesis

EPM310 is provided as an example of CBGA ester derivative obtainable by the following processes. The synthesis described in Examples 1 to 3 is directed to preparing EPM310 in three steps by first carboxylating of a commercial cannabigerol (CBG) to form cannabigerolic acid (CBGA), following by performing an esterification of the CBGA and then performing methylation reactions of the hydroxyl groups.

### Example 1. Synthesis of Cannabgerolic Acid (CBGA)

A mixture of Cannabigerol (CBG, 316 mg, 1 mmol) and 2 molar solution of magnesium methyl marbonate (MMC/2M, 1.5 ml, 3 mmol) in dimethylformamide (DMF) was heated at 130° C. for 3 hours. Then the reaction was cooled to 0° C., acidified with 10% hydrochloric acid and extracted with ether. The organic layer was washed with saline, dried over the drying agent magnesium sulfate (MgSO₄) and then evaporated. The crude compound was then cleaned by column chromatography (20% ether-petroleum ether).

### Example 2. Synthesis of Cannabigerolic Acid Methyl Ester (CBGA-ME)

To a solution of Cannabigerolic Acid (CBGA) (176 mg, 0.488 mmol) in 2.5 ml dichloromethane (CH₂Cl₂), was added 0.02 ml of methanol (CH₃OH, 0.488 mmol) and 7.2 mg of 4-Pyrrolidinopyridine (0.048 mmol). The reaction was stirred for 5 minutes at room temperature followed by the addition of the coupling agent, N,N′Dicyclohexylcarbodiimide (DCC) (121 mg, 0.585 mmol) and stirred overnight. Then the solvent was evaporated and the crude mixture acidified with 5% hydrochloric acid and extracted with dichloromethane (CH₂Cl₂). The organic layer was washed with saturated aqueous sodium bicarbonate (NaHCO₃), dried over the drying agent magnesium sulfate (MgSO₄) and then evaporated. The crude compound was then cleaned by column chromatography (2% ether-petroleum ether).

### Example 3. Synthesis of EPM310

To a mixture of CBGA-ME (374 mg, 1 mmol) and potassium carbonate (330 mg, 2.5 mmol) in dimethylformamide (DMF) (5 mL) is added methyl iodide (0.5 mL) and stirred overnight under a nitrogen atmosphere at rt. Then water (20 mL) is added and the resulting mixture was extracted with ether. The organic layer was neutralized, dried over drying agent magnesium sulfate (MgSO₄) and then evaporated. The crude compound is then cleaned by column chromatography (3-5% ether-petroleum ether) to isolate the title compound.

### Example 4. In-Vivo Model of Pain

The therapeutic effect of EPM310 on pain is examined in the experimental model of hot-plate test, measuring the analgesic activity of compound 1 based on the reaction time of mice to lick their forepaws and/or jump after being placed on an aluminum hot plate heated to, and maintained at about 55° C. as described in the U.S. Pat. No. 5,338,753, with minor modifications. An aluminum surface is maintained at about 55° C. by circulating water through the passages in the metal. A clear plastic cylinder is placed on the surface to prevent escape. The end point is reached when the mouse either performed a hind paw lick or jumped off the surface; in no case are the animals kept more than 30 seconds on the plate. Control values are measured 3 hours before the test values. Mice are treated with EPM310 at different doses ninety (90) minutes before the hot plate test. The percent change in response time (latency) is calculated by comparing the mean of the control values with the mean of the test values and statistical significance determined by a paired t test.

### Example 5. In-Vivo Model of Inflammatory

The therapeutic effect of EPM310 on inflammatory is examined in the experimental model of inflammation (paw edema) in arachidonic acid or platelet activating factor (PAF) injected mice, as described in the U.S. Pat. No. 5,338,753. Briefly, PAF (1.0 μg) or arachidonic acid (1.0 mg) dissolved in 50 μL of 5% ethanol in saline, is injected subcutaneously into the plantar surface of the right hind paw of ether-anesthetized CD-1 female mice (20-25 g). The volume of the right foot is measured to the level of the lateral malleous by water displacement before treatment, fifteen minutes after PAF injection, or thirty minutes after arachidonic acid injection. The mice are then treated with EPM310 injections at different doses. The change in paw volume is calculated for each mouse and the significance for each group is determined by a paired t test.

### Example 6. In-Vivo, Pentylenetetrazole-Induced (PTZ) Model of Generalized Seizures

The antiepileptic effects of EPM310 is tested in an acute experimental epilepsy model in pentylenetetrazole (PTZ) injected rats as described in Obay et. al. (Peptides, 2007, 28, 1214-1219). Briefly, adult male Wistar albino rats are divided into a control group, and four experimental groups with seven rats in each group. In order to generate epileptic seizures, PTZ (50 mg/kg) is injected intraperitoneally. The experimental groups receive intraperitoneal injections of EPM310 at different doses 30 min before PTZ injection. After PTZ injection, the rats are kept in cages and their behaviors are observed for 30 min. The latencies are separated into three components: first myoclonic jerk (FMJ), generalized clonic seizures (GCS) and tonic generalized extension (TGE).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method of treating a disease, disorder or symptom amenable to treatment with cannabigerol (CBG), the method comprising the step of administering to a subject in need thereof a therapeutically effective amount of a compound represented by the structure of Formula (I), 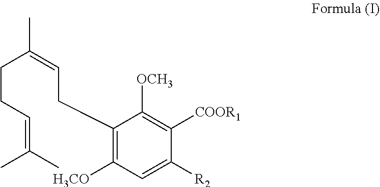
wherein
R₁ is selected from the group consisting of a hydrogen, a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
R₂ is selected from the group consisting of a linear or branched unsubstituted or substituted C₁-C₁₅ alkyl, a linear or branched unsubstituted or substituted C₂-C₁₅ alkenyl, and a linear or branched unsubstituted or substituted C₂-C₁₅ alkynyl;
and stereoisomers, geometrical isomers, and salts thereof.

2. The method according to claim 1, wherein the disease, disorder or symptom amenable to treatment with CBG is selected from the group consisting of Non-Alcoholic Fatty Liver Disease (NAFLD), chronic kidney disease (CKD), obesity, hyperglycemia, diabetes, metabolic syndrome and/or obesity related diseases.

3. The method according to claim 2, wherein the disease is obesity.

4. The method according to claim 1, wherein R₁ is a linear unsubstituted C₁-C₁₅ alkyl.

5. The method according to claim 1, wherein R₁ is methyl.

6. The method according to claim 1, wherein R₁ is hydrogen.

7. The method according to claim 1, wherein R₂ is a linear unsubstituted C₁-C₁₅ alkyl.

8. The method according to any one of claims 1-5, wherein R₂ is a linear unsubstituted C₅ alkyl.

9. The method according to claim 1, which is represented by the structure of Formula (II), 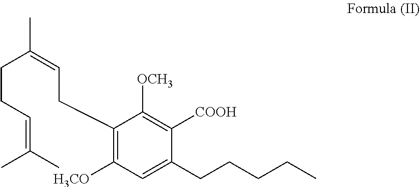 [SMILES: CCCCCc1cc(OC)c(C/C=C(/C)CCC=C(C)C)c(OC)c1C(=O)O]
and stereoisomers and salts thereof.

10. The method according to claim 1, which is represented by the structure of Formula (III), 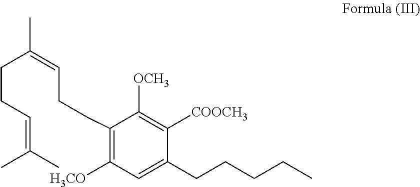 [SMILES: CCCCCc1cc(OC)c(CC/C=C(/C)CCC=C(C)C)c(OC)c1C(=O)OC]
and stereoisomers thereof.

11. The method according to claim 1, comprising the step of administering to the subject in need thereof a pharmaceutical composition comprising a therapeutically effective amount of the compound represented by the structure of Formula (I) and a pharmaceutically acceptable carrier or excipient.

12. The method according to claim 11, wherein the pharmaceutical composition further comprises at least one additional active ingredient, wherein the additional active ingredient comprises at least one additional cannabinoid compound.

13. The method according to claim 12, wherein the additional cannabinoid compound comprises a cannabigerolic acid (CBGA) ester represented by Formula (IV), a cannabinolic acid (CBNA) ester represented by Formula (V), a cannabidiolic acid (CBDA) ester represented by Formula (VI), a tetrahydrocannabinolic acid (THCA) ester represented by Formula (VII) or any combination thereof, 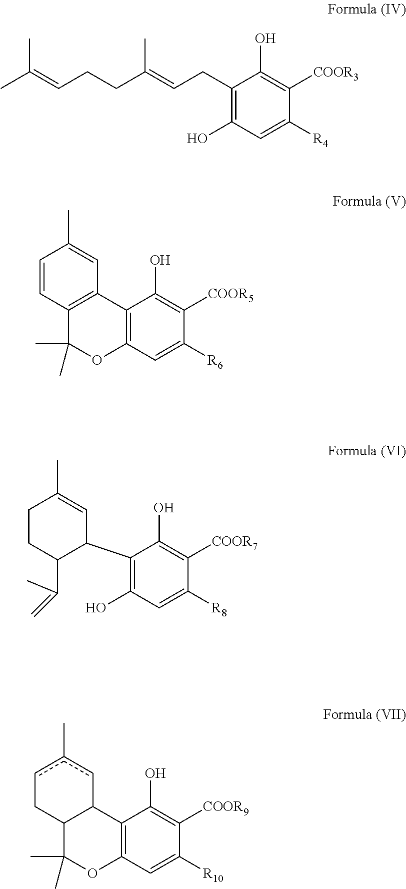
wherein
each of R₃, R₄, R₅, R₆, R₇, R₈, R₉ and R₁₀ is independently selected from the group consisting of a linear or branched, unsubstituted or substituted C₁-C₁₅ alkyl, a linear or branched, unsubstituted or substituted C₂-C₁₅ alkenyl, and a linear or branched, unsubstituted or substituted C₂-C₁₅ alkynyl;
one of the dashed lines in Formula (VII) represents a double bond and the other dashed line represents a single bond;
and stereoisomers and salts thereof.

14. The method according to claim 11, wherein the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a preservative, an antioxidant, a flavoring agent, a colorant, and a mixture or combinations thereof.

15. The method according to claim 11, a form selected from the group consisting of tablet, pill, capsule, pellets, granules, powder, a wafer, coated or uncoated beads, lozenge, sachet, cachet, elixir, a depot system, an a patch, suspension, dispersion, emulsion, solution, syrup, aerosol, oil, ointment, suppository, a gel, and a cream.

16. The method according to claim 12, wherein the additional cannabinoid compound is present in one or more extracts of a cannabis plant.

17. The method according to claim 16, wherein the cannabis plant extract is obtained from a strain selected from the group consisting of Cannabis sativa, Cannabis indica, Cannabis ruderalis, a hybrid strain, and combinations thereof.

18. The method according to claim 16, wherein the cannabis plant extract is obtained from a strain selected from the group consisting of a high-CBD strain, a high-THC strain, and a combination thereof.

19. The method according to claim 16, wherein the cannabis plant extract comprises at least one cannabinoid selected from the group consisting of cannabidiol (CBD), tetrahydrocannabinol (THC), cannabinol (CBN), cannabigerol (CBG), cannabichromene (CBC), thereof and combinations thereof.
